(19) United States
(12) Patent Application Publication
(10) Pub. No.: US 2026/0167915 A1
(43) Pub. Date: Jun. 18, 2026

(54) SYSTEM AND METHOD FOR EX-VIVO DEVELOPMENT

(71) Applicant: Becoming Bio, Inc., San Francisco, CA (US)

(72) Inventor: Jack Andrew Cohen, San Francisco, CA (US)

(73) Assignee: Becoming Bio, Inc., San Francisco, CA (US)

(21) Appl. No.: 19/423,617

(22) Filed: Dec. 17, 2025

Related U.S. Application Data
(60) Provisional application No. 63/734,831, filed on Dec. 17, 2024.

(51) Int. Cl. C12M 3/00; C12M 1/00; C12M 1/32; C12M 1/34; C12M 1/36

(52) U.S. Cl. CPC C12M 23/44; C12M 23/12; C12M 29/00; C12M 41/34; C12M 41/44; C12M 41/48

(57) ABSTRACT

The system can include: a set of incubation modules, a fluid module, a gas module, a sensor module, and a processing system. The method can include incubating a set of biological specimens using the system. The system and/or method can function to incubate (e.g., gestate) a set of biological specimens (e.g., embryos) from a one-cell stage through full development or any range therebetween. The set of biological specimens can optionally be used for therapeutics.

### CROSS-REFERENCE TO RELATED APPLICATIONS

This application claims the benefit of U.S. Provisional Application number 63/734,831 filed 17 Dec. 2024, which is incorporated in its entirety by this reference.

### TECHNICAL FIELD

This invention relates generally to the embryo development field, and more specifically to a new and useful system and method in the embryo development field.

### BRIEF DESCRIPTION OF THE FIGURES

FIG. 1 is a schematic representation of a variant of the system.

FIG. 2 is a schematic representation of a variant of the method

FIG. 3 is a schematic representation of an example of the system

FIG. 4A depicts an example of an incubation module.

FIG. 4B depicts another example of an incubation module.

FIG. 5 depicts a specific example of the system.

FIGS. 6A-6C are schematic representations of examples of determining parameter values.

FIG. 7 is a schematic representation of an example of the system.

FIGS. 8A-8M are schematic representations of specific examples of system components.

FIGS. 9A-9L depict examples of the system.

### DETAILED DESCRIPTION

The following description of the embodiments of the invention is not intended to limit the invention to these embodiments, but rather to enable any person skilled in the art to make and use this invention.

### 1. Overview

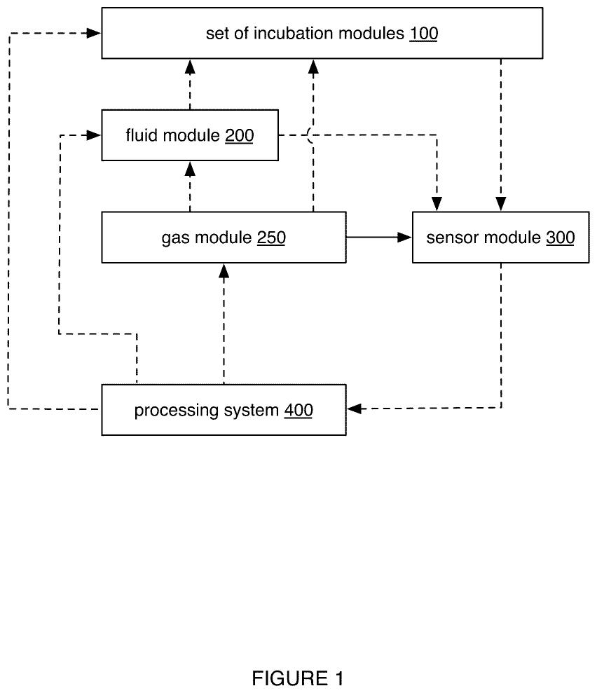

As shown in FIG. 1, the system can include: a set of incubation modules 100, a fluid module 200, a gas module 250, a sensor module 300, and a processing system 400. However, the system can additionally or alternatively include any other suitable components.

In variants, the system can function to incubate (e.g., gestate) a set of embryos from a one-cell stage through full development or any range therebetween. Additionally or alternatively, the system can function to incubate any other biological specimen. The embryos and/or other biological specimens can optionally be used for therapeutics.

In an example, the system (e.g., incubation machine, gestation machine, metabolic exchange machine, etc.) can include one or more incubation modules, where each incubation module houses at least one embryo. An incubation module can include: a well; gel within the well, where an embryo is positioned on or within the gel (e.g., on the gel surface, within a gel cavity, etc.); a uterine circuit configured to supply media to the well; and a placental circuit including a channel configured to flow media (e.g., the same or different from the well media) beneath the well. An opening (e.g., pore, mesh, etc.) in the base of the well (beneath the embryo) can overlap with an opening in the roof of the placental circuit channel, such that the placenta of the embryo can grow into the openings and access media in the channel. In a specific example, flow of media through the channel can be controlled using a pressure column system, including a first reservoir of media above the channel inlet, a second reservoir of media above the channel outlet, a first sensor (e.g., dielectric sensor) measuring the quantity of media in the first reservoir, a second sensor (e.g., dielectric sensor) measuring the quantity of media in the second reservoir, and a set of pumps to transport media to and from the reservoirs based on the sensor measurements. Incubation parameters (e.g., media composition, gas composition, flow rate, etc.) can optionally be controlled in real-time in response to measurements acquired by sensors. In a specific example, determining parameter values can include: acquiring a set of images of an embryo using an imaging device (e.g., light sheet microscope), determining a 3D model of the embryo based on the set of images, determining a developmental stage of the embryo, and determining parameter values based on the developmental stage.

However, the system and/or method can be otherwise configured.

### 2. Technical Advantages

Variants of the technology can confer one or more advantages over conventional technologies.

First, variants of the technology can enable extrauterine gestation up to and/or beyond placentation (e.g., beyond stage E11.5, beyond stage E12.5, through full gestation, etc.). In an example, the biological specimen can be gestated through development of early organ tissue—including multiple cell types and initial organ structure. This tissue (e.g., optionally aggregated from multiple biological specimens) can be used as a therapeutic. In an example, the system can support high-throughput gestation of a set of biological specimens (e.g., at least 10, at least 20, at least 50, etc.).

Second, variants of the technology can include at least two fluid circuits: a uterine circuit supplying fluid to the well housing the embryo (e.g., simulating the amniotic fluid), and a maternal circuit supplying fluid to the placenta (e.g., simulating the maternal blood supply). In a specific example, the maternal circuit can include a channel located beneath the well, wherein an opening in the well base and a corresponding opening in the channel roof allows for the placenta to access the fluid in the channel. Variants of the technology can include endothelial cells within, on, or near the maternal circuit channel to encourage placental growth towards the channel. Variants of the technology can control flow through the maternal circuit channel using a gravity-based system, where the amount of fluid in a first reservoir connected to the channel inlet and the amount of fluid in a second reservoir connected to the channel outlet prescribes a pressure differential between the channel inlet and channel outlet. In variants, this gravity-based system can enable precise control of the fluid in the channel.

Third, variants of the technology can adjust environmental parameter values (e.g., temperature, gas composition, etc.), media parameter values (e.g., glucose concentration, etc.), and/or other protocol values in response to sensor measurements, including images acquired of the developing biological specimen. For example, the system can provide real-time feedback, enabling a fully or partially autonomous incubation machine (e.g., gestation machine). In a specific example, the parameter values can be controlled using a protocol model, trained output parameter values that are optimized to minimize the difference between the (current) ex-utero biological specimen to a (target) in-utero biological specimen at the same developmental stage.

Fourth, current methods of developmental stage evaluation rely on experts to manually assess a developing biological specimen, which hinders automation and high-throughput analysis. Variants of the technology can include a trained machine learning model which can output a biological specimen's developmental stage (e.g., Theiler Stage) and optionally an associated confidence level based on a set of images acquired of the biological specimen. This classification can optionally be encoded as an embedding, which can be used for training data augmentation (e.g., interpolating between embeddings and optionally decoding to generate artificial images); used as an input for a protocol model; and/or otherwise used.

However, further advantages can be provided by the system and method disclosed herein.

### 3. System and/or Method

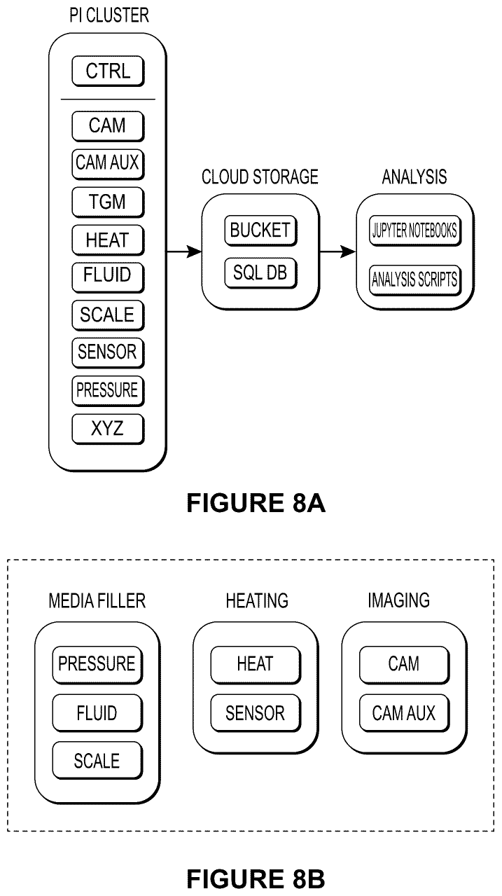

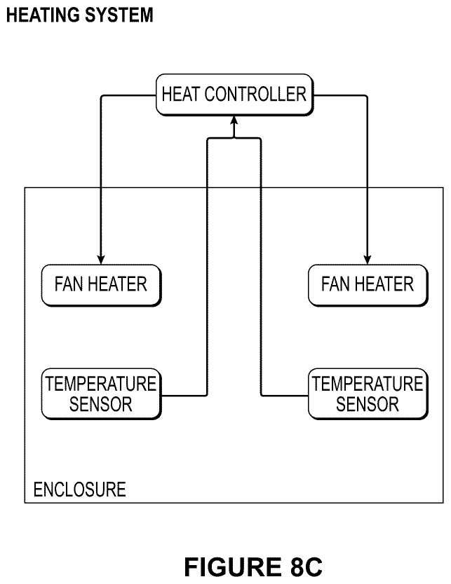

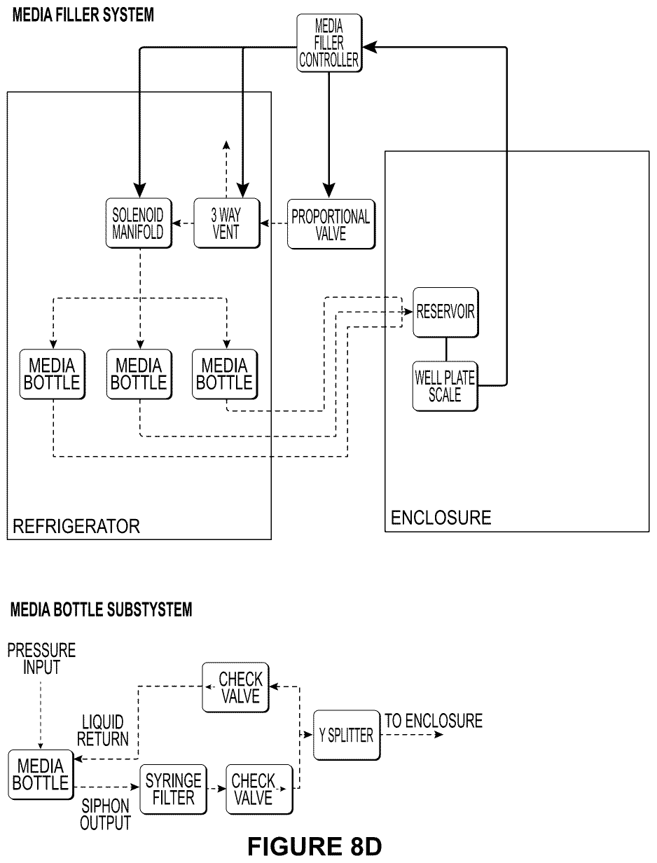

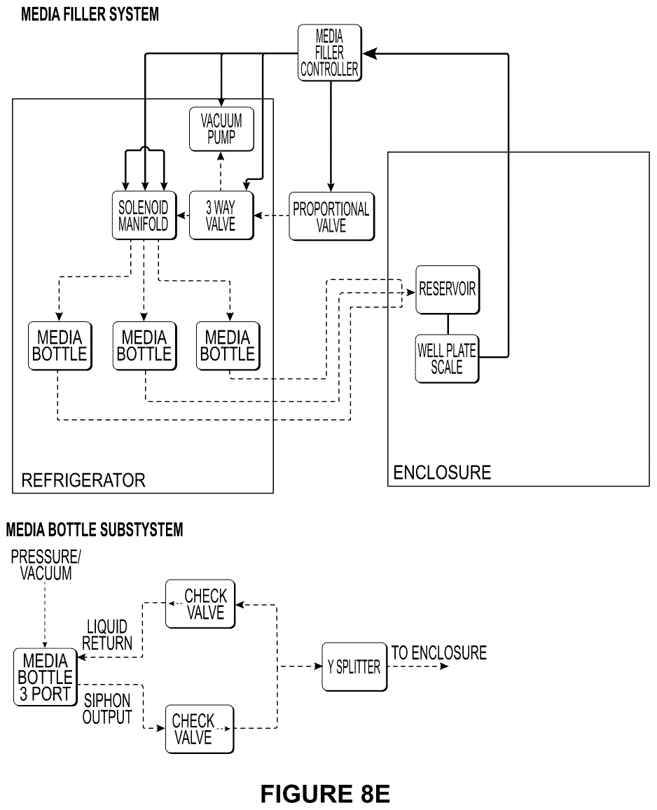

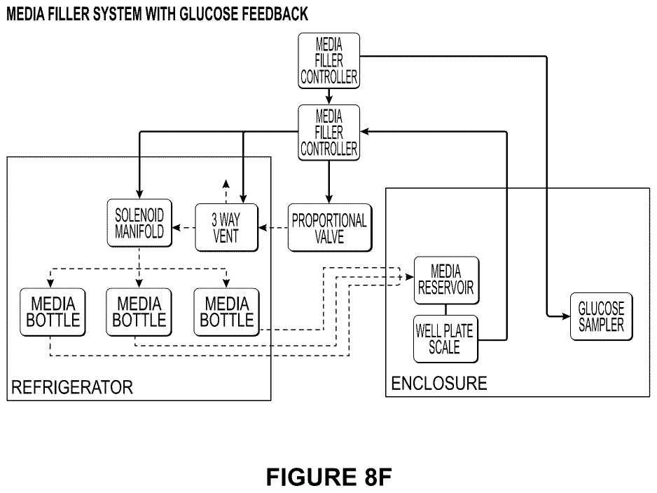

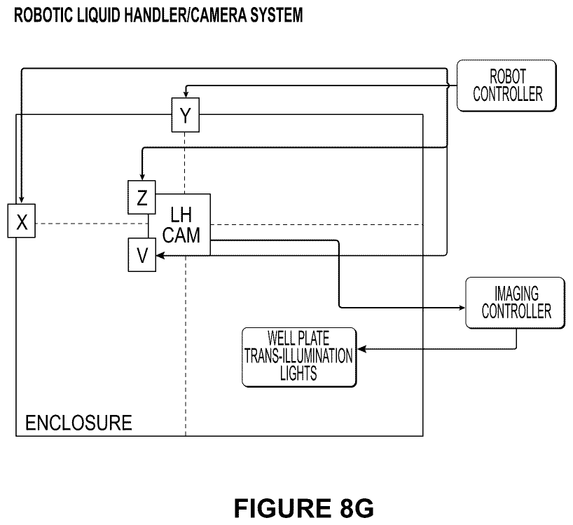

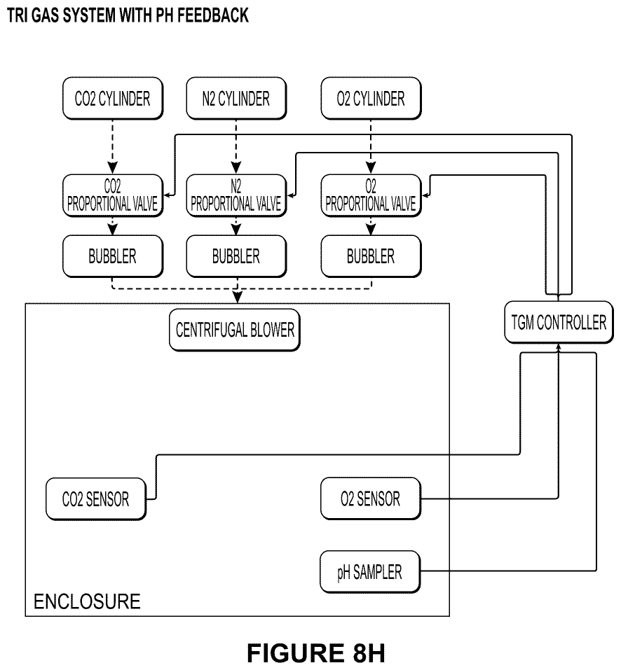

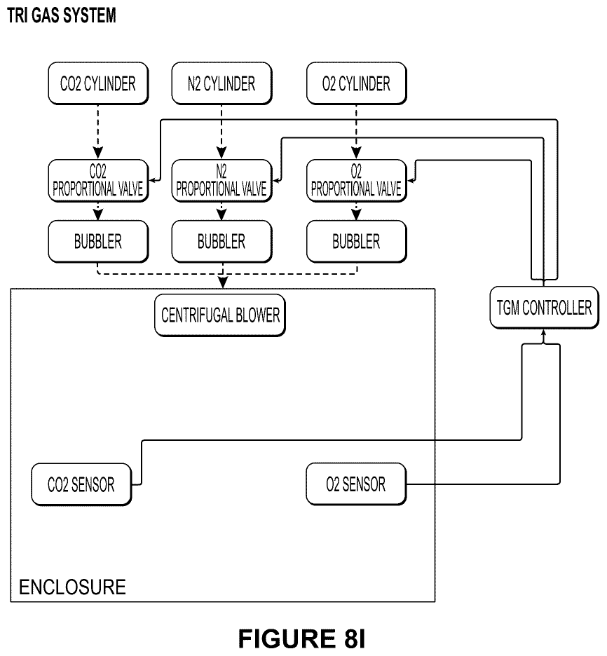

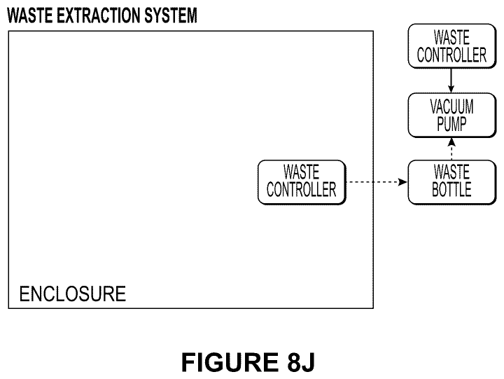

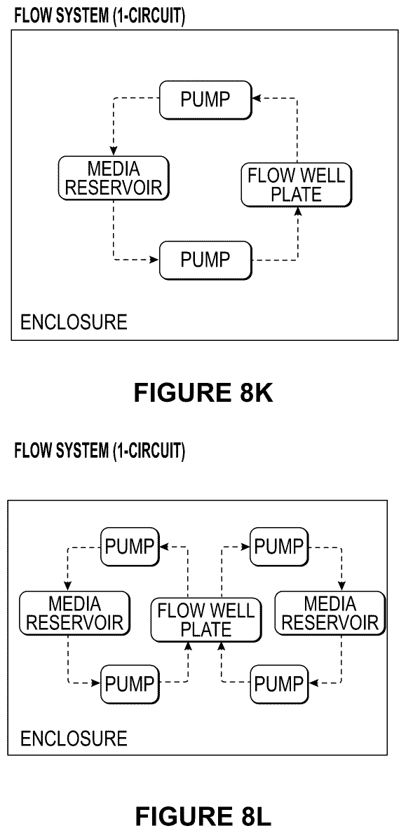

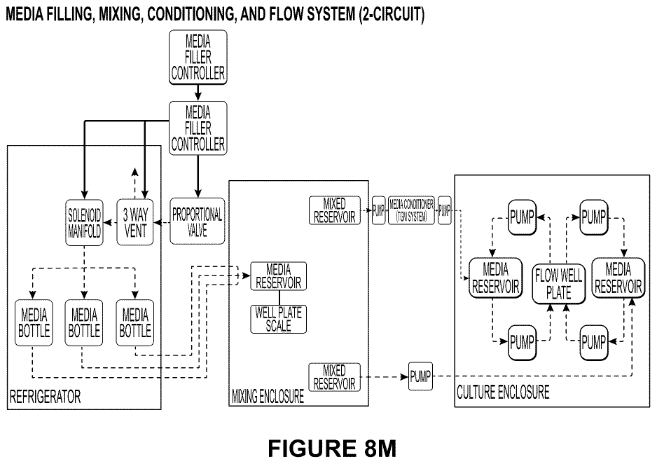

As shown in FIG. 1, the system can include: a set of incubation modules 100, a fluid module 200, a gas module 250, a sensor module 300, and a processing system 400. However, the system can additionally or alternatively include any other suitable components. Specific examples of components are shown in FIG. 8A-8M.

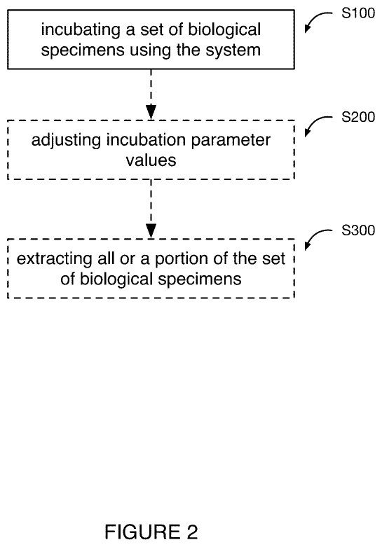

As shown in FIG. 2, the method can include incubating a set of biological specimens using the system S100. The method can optionally include adjusting parameter values during incubation S200, extracting all or a portion of the set of biological specimens S300, and/or any other suitable steps. As used herein, incubating a biological specimen can optionally refer to: culturing, developing, maturing, and/or gestating the biological specimen.

The system can be used with a set of biological specimens. The number of biological specimens in the set can be 1-1000 or any range or value therebetween (e.g., 1-32, 1-128, at least 2, at least 10, etc.), but can alternatively be greater than 1000. The biological specimens can be human, mouse, any other animal species (e.g., rat, guinea pigs, sheep, monkeys, chimpanzee, ape, etc.), and/or derived from any other organism. The biological specimens preferably include embryos, but can additionally or alternatively include fetuses, stem cells, any organism, and/or any other biological material. In variants, the system can function to incubate a set of biological specimens from a one-cell stage (e.g., fertilized embryo, stem cell, etc.) through full development or any range therebetween (e.g., E0.5-E9.5; E0.5-P0, beyond E11.5, beyond E12.5, up to and/or beyond placentation, 0-8 weeks, 0-12 weeks, etc.).

The set of incubation modules 100 function to house the set of biological specimens and provide nutrients (e.g., media) to the set of biological specimens. In a first variant, each incubation module can house a single biological specimen. In a second variant, each incubation module can house multiple biological specimens (e.g., at least 2 biological specimens, at least 3 biological specimens, at least 4 biological specimens, at least 5 biological specimens, etc.).

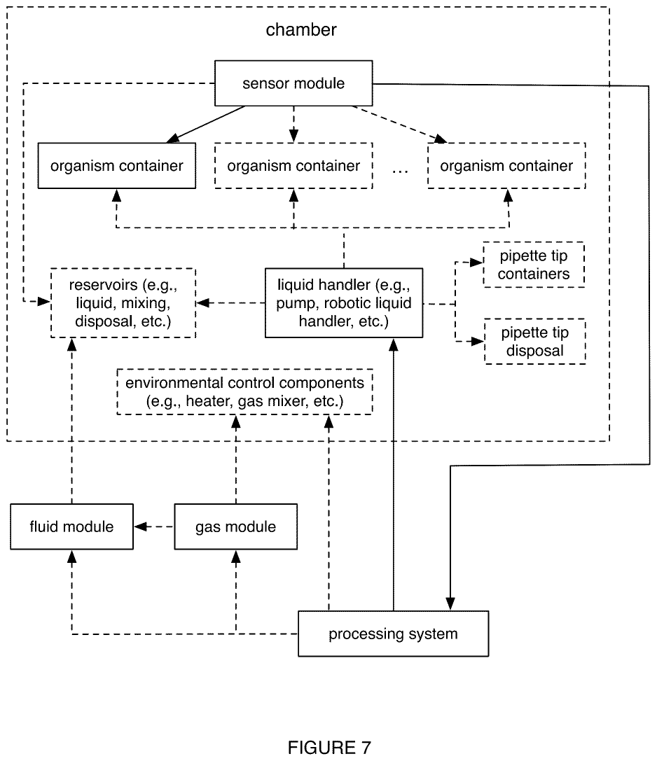

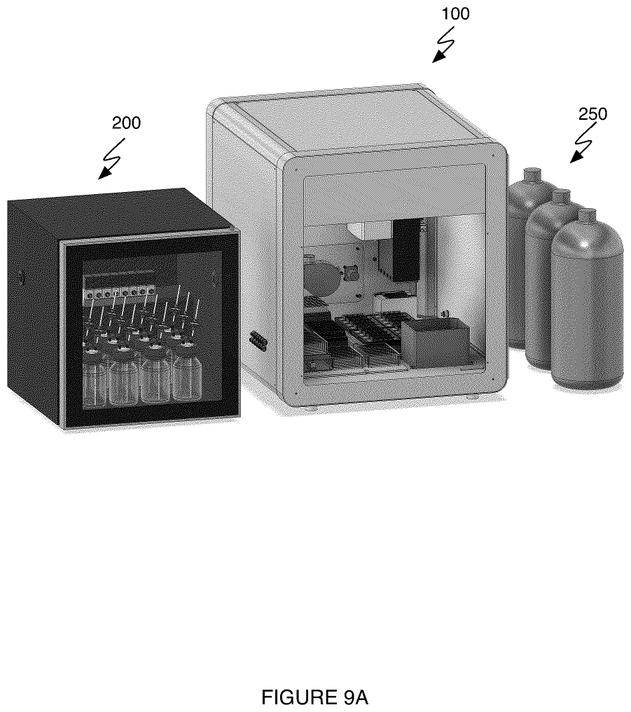

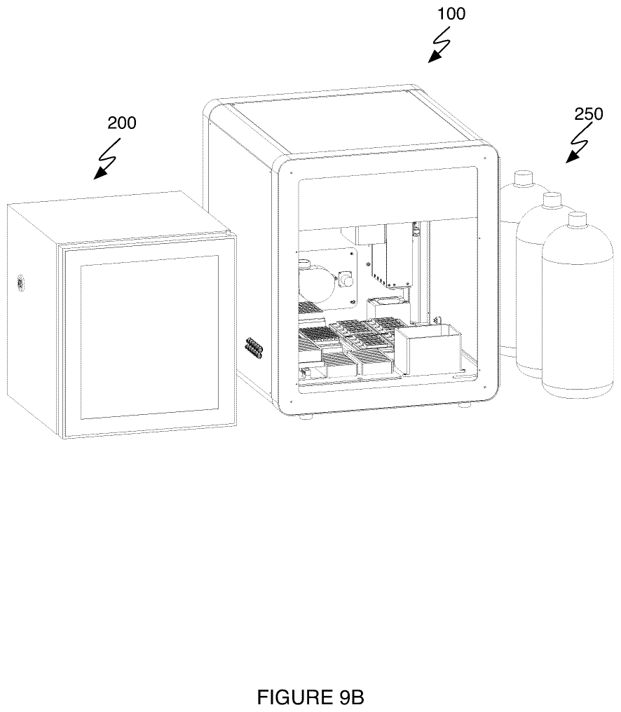

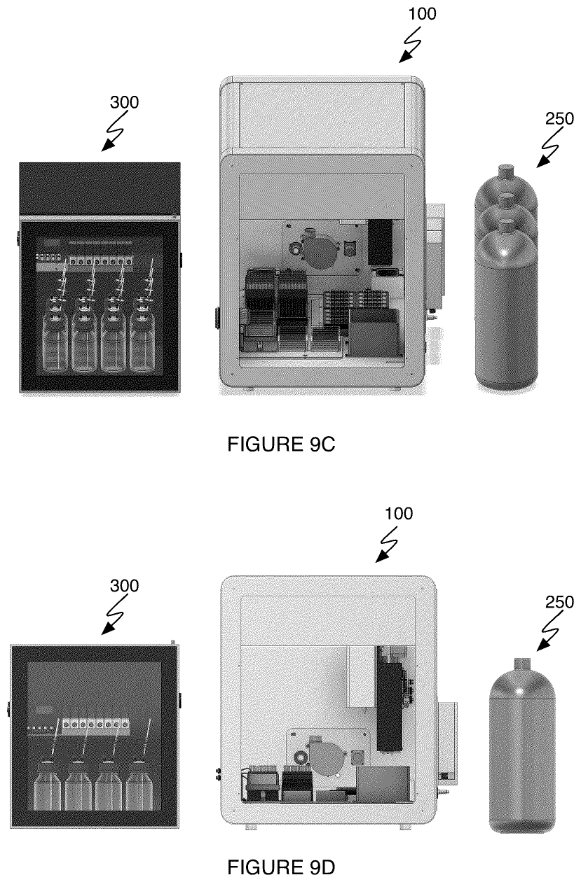

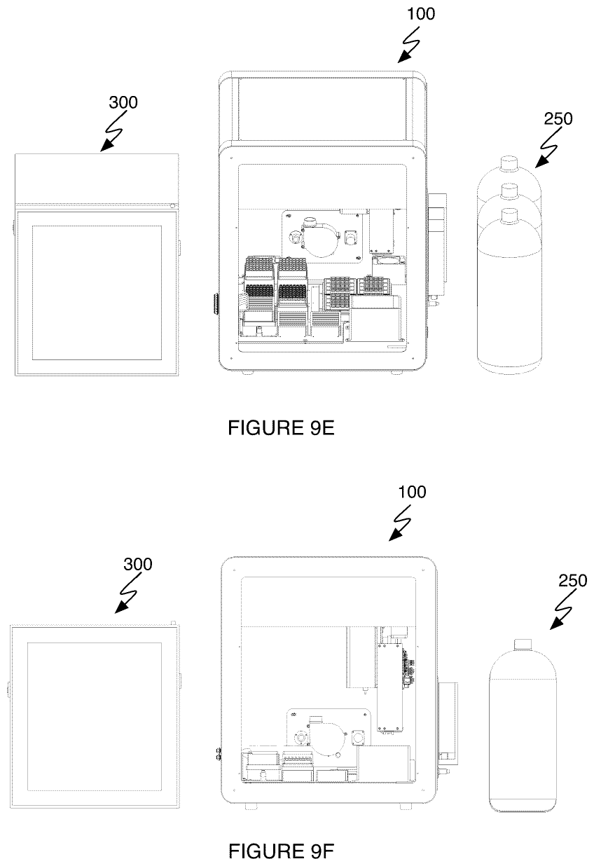

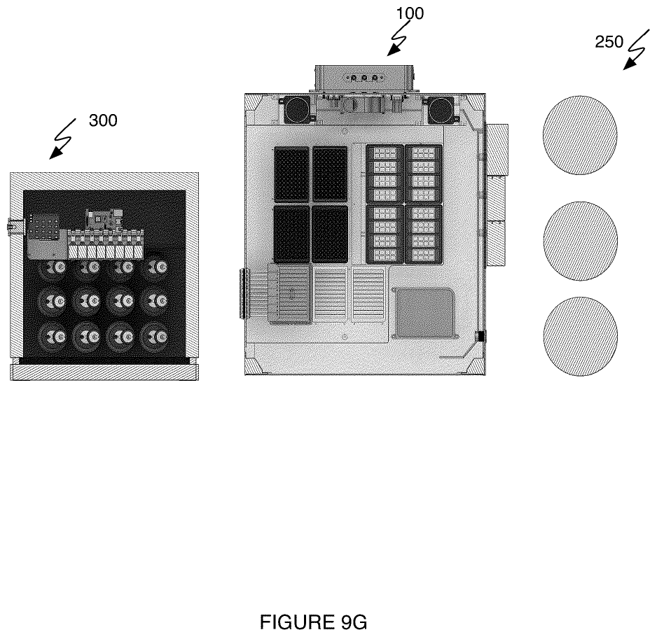

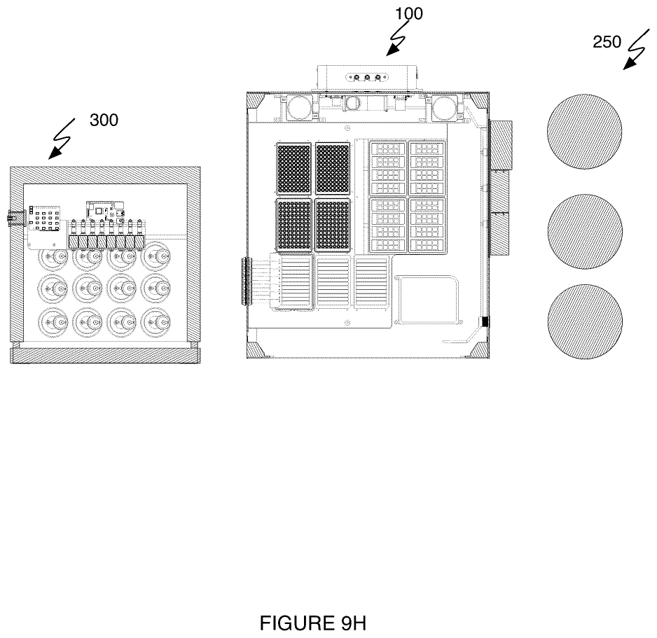

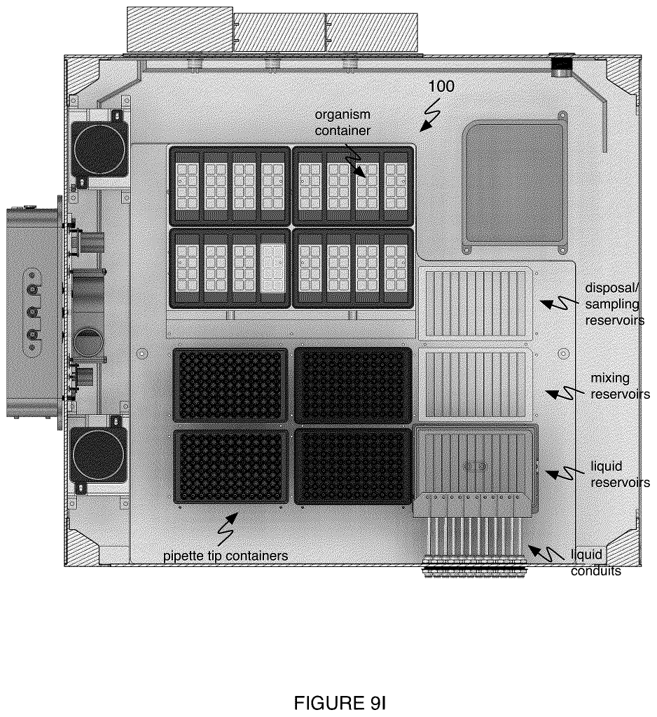

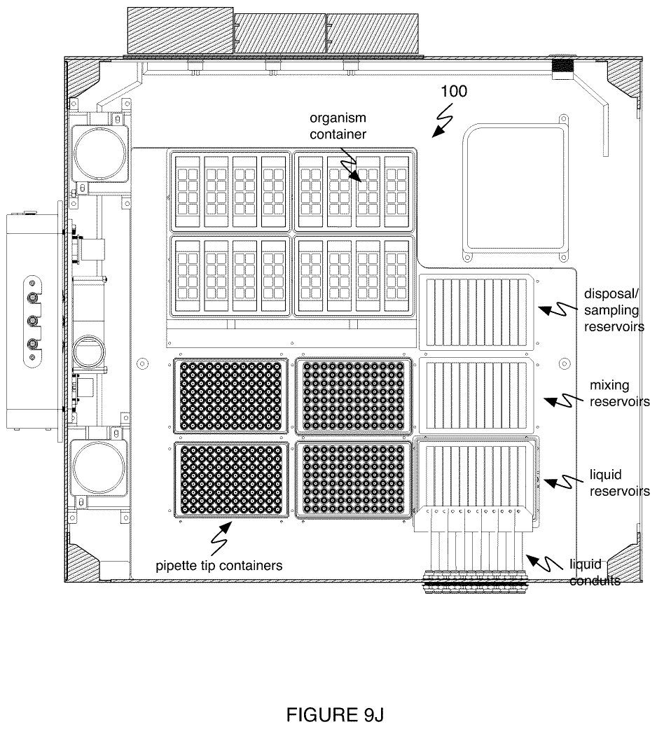

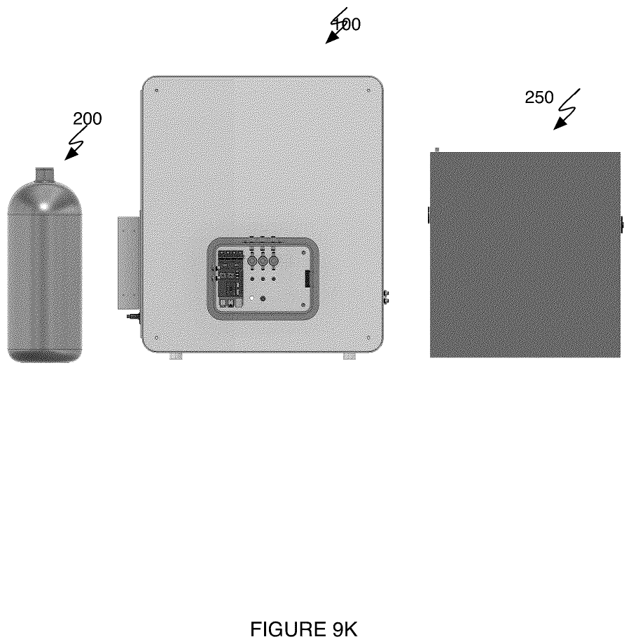

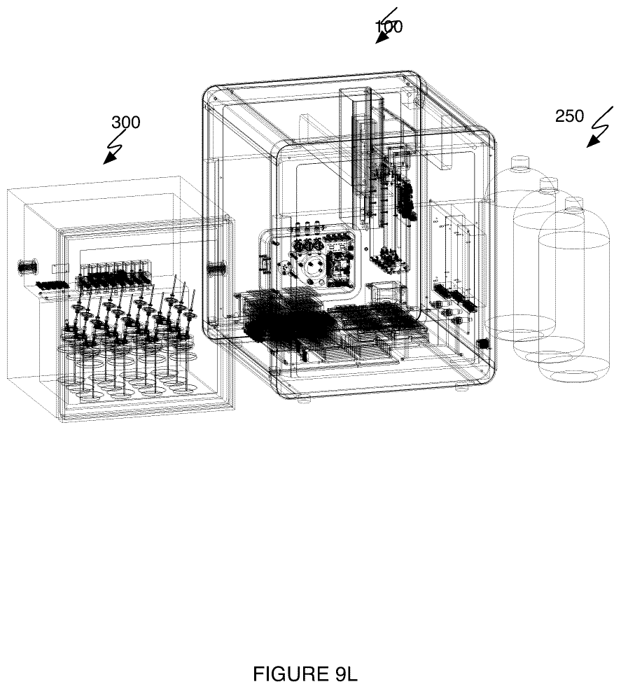

The set of incubation modules 100 can optionally be contained within a chamber (e.g., an incubation chamber), which can function to incubate the set of biological specimens. Examples are shown in FIG. 7, and FIGS. 9A-9D. The chamber can include a single enclosure and/or multiple enclosures. The enclosure(s) can optionally be insulated and/or shielded (from light).

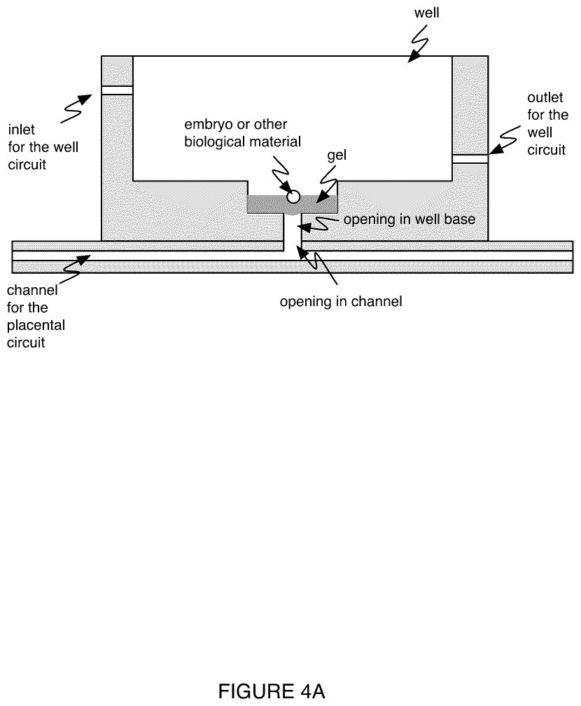

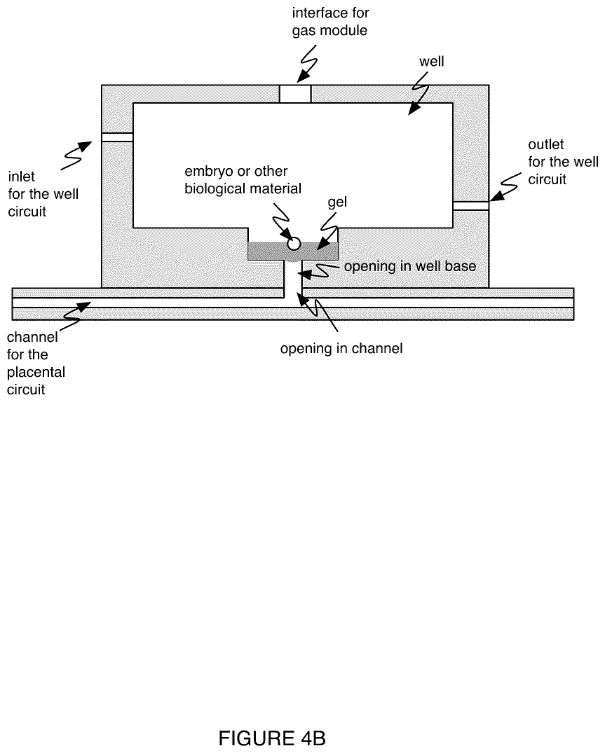

Each incubation module can include a container configured to house (e.g., contain) one or more biological specimens. Examples are shown in FIG. 4A and FIG. 4B. For example, the container can be a well (e.g., a perfusion well). In an example, the set of incubation modules 100 can include a set of containers. The set of containers can be arranged in an array (e.g., a 3D array, a horizontal 2D array, a vertical 2D array, a vertical or horizontal linear array, a set of offset vertical linear arrays, a set of offset horizontal linear arrays, etc.). The container is preferably static, but can alternatively be actuated. The container preferably does not include a roller culture system, but can alternatively include a roller culture system. The container can be between 10 mm³-50 cm³ (e.g., 15 mm³-60 mm³, 20 mm³-240 mm³, 50 mm³-1000 mm³, 100 mm³-10 cm³, etc.), but can alternatively be less than 10 mm³ or greater than 50 cm³. Containers can optionally be connected to one another (e.g., connected fluid circuits, sharing media, connected gas circuits, sharing gas, etc.). The container can include one or more interfaces for the fluid module 200 (e.g., one or more inlets and outlets) and/or one or more interfaces for the gas module 250. In a first example, the container can be open (e.g., open to air within a chamber housing the container); an example is shown in FIG. 4A. In a second example, the container can be closed (e.g., where the container includes an interface for the gas module 250); an example is shown in FIG. 4B.

The container can optionally include gel surrounding all or a portion of the biological specimen. For example, the gel can be disposed between the biological specimen (e.g., embryo) and the base surface of the well. In a specific example, the placenta of an embryo specimen can extend (e.g., grow) into the gel (e.g., downwards). Examples of the gel can include Matrigel, PEG-based hydrogels, and/or any other suitable gels. The gel can optionally contain one or more of: fibronectin, laminin, heparan sulfate, human skeletal growth factor, nutrients, and/or any other additives. The gel can optionally be replenished and/or replaced during incubation. The amount of gel in the container can be between 1 μL-100 mL or any range or value therebetween (e.g., 10 μL-30 μL, approximately 15 μL, less than 50 μL, greater than 5 μL, etc.), but can alternatively be less than μL or greater than 100 mL. The base of the well can optionally include a recess, wherein the gel is located within the recess. The gel can be contained entirely within the recess or can extend beyond the recess. In variants, this recess can protect the biological specimen from fluid flow in the well. A porous membrane can optionally be positioned within the container over the biological specimen (e.g., over the gel and the biological specimen). In variants, this can constrain the biological specimen (e.g., such that it is not disturbed by fluid flow in the container).

In a first variant, the surface of the gel can be approximately flat (e.g., parallel to the well base), wherein the biological specimen (e.g., a human embryo) can be positioned on the gel surface at the start of incubation. In a second variant, the surface of the gel can include one or more valleys (e.g., low points), wherein the biological specimen (e.g., mouse embryo, human embryo, etc.) can be positioned in a valley (e.g., at the nadir of a valley). In a first example, the valley can be a cavity (e.g., a uterine cavity, a crypt, etc.) for the biological specimen (e.g., a mouse embryo), wherein the gel supports the biological specimen from the base and all or a portion of the sides of the embryo. In a second example, the valley can be a concave arc, wherein the biological specimen is positioned at the base of the arc. The gel surface can optionally include a single valley or a set of multiple valleys. In a first example, the gel surface can include a single valley, where a single biological specimen is positioned in the valley. In a second example, the gel surface can include multiple valleys, where each single biological specimen in a set of biological specimens (e.g., a subset of the set of biological specimens) is positioned in a valley.

The gel can optionally be a cross-linkable gel. In an example, the gel can be deposited into the container as a liquid and then cross-linked (e.g., via UV light). In a first example, one or more valleys (e.g., crypts) in the gel can be formed using photoablation. In a second example, one or more valleys in the gel can be formed by masking the gel during cross-linking to leave the portion of the gel in the valley(s) uncured (e.g., uncross-linked), wherein the uncured gel can be removed. In a third example, one or more valleys in the gel can be formed by placing a mold (e.g., template) for the valley(s) into the uncured gel during cross-linking. In a fourth variant, the valley can be formed by removing cured (e.g., cross-linked) gel (e.g., by cutting into the gel).

The biological specimen (e.g., a mouse embryo) can optionally be transferred to the gel (e.g., placed on the surface of the gel) with a specific orientation. However, the biological specimen (e.g., a human embryo) can alternatively not be transferred with a specific orientation. For example, an embryo can be placed within a valley of the gel (e.g., the uterine crypt) such that the extra embryonic side (e.g., proximal side) of the embryo is at the base of the valley. The biological specimen can optionally be transferred to the gel using a robotic system. The biological specimen can optionally be transferred to the gel under infrared guidance. In variants, infrared light can enable the valley to be better viewed.

In a first variant, the incubation module includes static media (e.g., surrounding the biological specimen, surrounding the gel, etc.). In an illustrative example, the system can include a set of wells holding media, each well housing one or more biological specimens in the set of biological specimens.

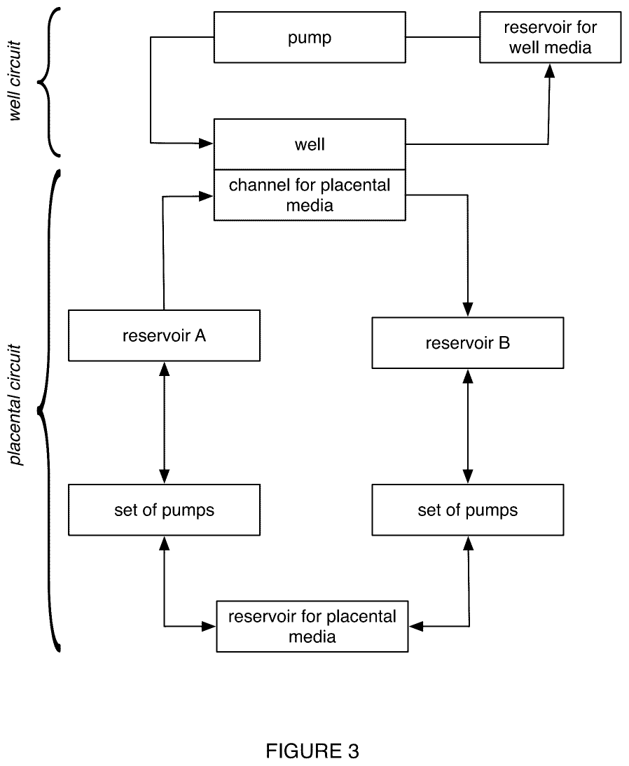

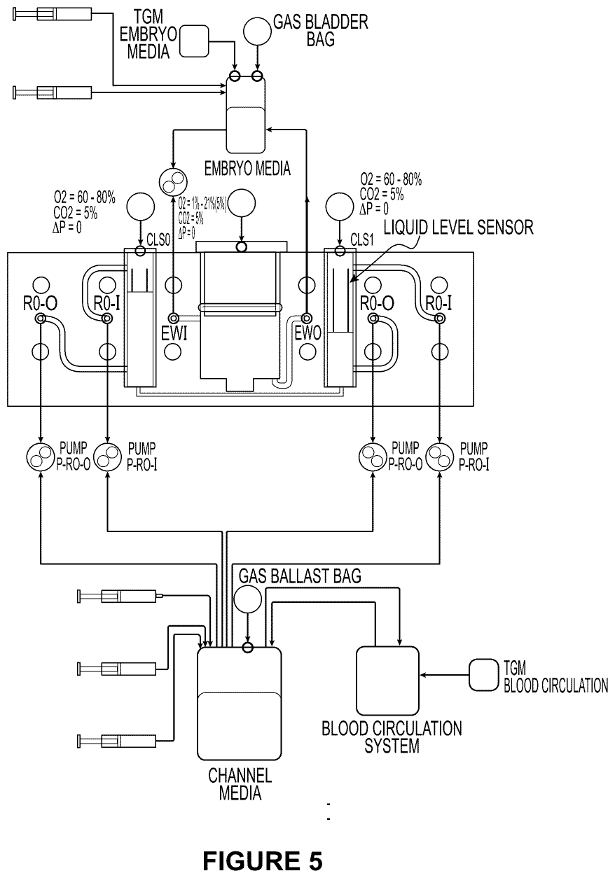

In a second variant, the incubation module can include or interface with one or more fluid circuits supplying media to the container (e.g., where media flows into the container via an inlet and out of the container via an outlet). Examples are shown in FIG. 3, FIG. 5, FIG. 8K, FIG. 8L, and FIG. 8M. The number of fluid circuits can be 1-4 or any range or value therebetween (e.g., 1, 2, 3, 4, at least 2, at least 3, etc.), but can alternatively be greater than 4. Flow through the fluid circuit(s) can be controlled by the fluid module 200 and/or by the processing system 400.

In a first embodiment, a fluid circuit (e.g., well circuit, uterine circuit, etc.) can supply media to the container (e.g., above the gel). In an illustrative example, this fluid circuit can supply fluid surrounding the embryo (e.g., simulating the uterine nutrient supply in amniotic fluid). In an example, the container (e.g., well) can include an inlet and an outlet, where the fluid circuit supplies media into the container via the inlet, and removes media from the container via the outlet. In a specific example, the inlet and outlet can be located on opposing sides of the container. The inlet location preferably at a greater height than the location of the biological specimen (e.g., at least 0.5 mm, at least 1 mm, at least 2 mm, at least 5 mm, etc.). In variants, this can shield the biological specimen from fluid flow (e.g., turbulence, drag, etc.) that could interfere with development and/or could move the biological specimen. A baffle can optionally additionally or alternatively be used to shield the biological specimen from fluid flow. The fluid circuit can optionally supply media to the container using pulsatile flow. The fluid circuit can optionally periodically reverse flow (e.g., flowing into the outlet, and out of the inlet). In variants, pulsatile flow and/or reversing flow can mitigate stagnant points in the container.

In a second embodiment, a fluid circuit (e.g., gel circuit) can provide media to the gel via one or more channels entering the gel (e.g., wherein nutrients diffuse through the gel to the biological specimen). The channel(s) preferably enter the gel (e.g., the edge of the gel) from the top, but can additionally or alternatively enter the gel from the sides and/or from below. The gel can optionally include interstitial channels to facilitate flow through the gel.

In a third embodiment, a fluid circuit (e.g., placental circuit, maternal circuit, etc.) can provide media adjacent to (e.g., beneath) the biological specimen, wherein the placenta can interface with the media in this fluid circuit. In an illustrative example, this fluid circuit can perfuse the placenta (e.g., simulating the maternal blood supply). The fluid circuit can include a channel where media flows through the channel. The channel is preferably located beneath the biological specimen (e.g., beneath the base of the container), but can additionally or alternatively be located on the side of the biological specimen and/or above the biological specimen. The channel can optionally be coupled to the base of the container. In a first specific example, the top surface (e.g., roof) of the channel can be the base surface (e.g., floor, bottom surface, etc.) of the container. In a second specific example, the top surface of the channel can abut the base surface of the container. The channel can optionally include an opening. For example, the channel can include an opening in the top surface (e.g., roof) of the channel (e.g., the top surface of the channel defines an opening). The container can optionally include an opening. For example, the container can include an opening in the base surface (e.g., floor) of the container (e.g., the base surface of the container defines an opening). The channel opening and/or the container opening can be a pore (e.g., through-hole), a slot, a porous mesh, a channel (e.g., a vertical channel), and/or any other opening. The gel in the container preferably does not extend into the channel through the opening(s), but can alternatively extend into the channel. In a first example, the bottom surface of the gel does not extend into the container opening. In a second example, the gel can extend into at least a portion of the container opening. The opening in the container preferably partially overlaps with the opening in the channel, but can alternatively not overlap. When the top surface of the channel is the bottom surface of the container, the opening in the channel can optionally be the opening in the container. When the gel surface includes a valley, an axis extending vertically downward from the valley (e.g., the nadir of the valley) can optionally intersect with the container opening and/or the channel opening. In variants, the opening(s) can enable the placenta to receive nutrients from fluid in the channel. In a specific example, the placenta can grow into the opening(s) to interface with fluid in the channel. The fluid circuit can optionally include endothelial cells (e.g., lining all or a portion of the channel and/or channel opening, within the channel and/or channel opening, near the channel and/or channel opening, etc.). In variants, these endothelial cells can provide signaling molecules which can encourage placenta growth towards the channel. Additionally or alternatively, the fluid circuit can include molecules secreted by endothelial cells (e.g., lining all or a portion of the channel and/or channel opening, within the channel and/or channel opening, near the channel and/or channel opening, etc.)

In a fourth embodiment, a combination of the previous embodiments can be used. The media preferably has a different composition across different fluid circuits, but can alternatively have the same composition. In an example, a first media (e.g., placental media) can flow through the placental circuit, and a second media (e.g., uterine media, well media, etc.) can flow through the uterine circuit (e.g., well circuit). In an example, an incubation module can include: a well having a base surface defining a first opening, wherein a biological specimen (e.g., embryo) is housed within the well; a gel, wherein the gel is disposed between the biological specimen and the base surface of the well; a first fluid circuit configured to supply a first media to the well; and a second fluid circuit including a channel coupled to the base surface of the well, wherein a second media passes through the channel.

However, the set of incubation modules 100 can be otherwise configured.

The fluid module 200 (e.g., liquid module) functions to control flow through fluid circuits, control fluid composition (e.g., media composition), and/or provide media and/or components therein to the set of incubation modules 100 (e.g., via one or more fluid circuits). The fluid module 200 can interface with the set of incubation modules 100 (e.g., with one or more fluid circuits), the gas module 250, the sensor module 300, and/or any other system component. In an example, the fluid module 200 can include one or more: liquid containers, reservoirs, liquid conduits (from the liquid containers to a liquid reservoir), filters, valves (e.g., three-way valves, solenoid manifolds, etc.), environmental control components (e.g., refrigeration system), pumps, a computing system (e.g., the processing system 400 or a separate system), and/or any other system components. In a specific example, the fluid module 200 can maintain the temperature of the liquids at a target refrigeration temperature (e.g., 0° C.-10° C., 4° C., etc.). The fluid module 200 can optionally contain media and/or media components, such as: basal media, human serum, water, glucose solution, DMEM, fetal bovine serum, rat serum, hormones, cytokines, growth factor, EBC, VEGF, PDGF, water, PBS, and/or any other media and/or media components. In a specific example, media (e.g., media in a uterine circuit, media in a gel circuit, etc.) can include less than a threshold percentage of rat serum (e.g., less than 98% rat serum, less than 95% rat serum, less than 90% rat serum, less than 80% rat serum, less than 70% rat serum, less than 60% rat serum, less than 50% rat serum, etc.). In a specific example, media (e.g., media in a uterine circuit and/or a gel circuit) can include less than a threshold concentration of alpha-2-Macroglobulin (e.g., less than 100 μg/ml, less than 90 μg/ml, less than 80 μg/ml, less than 70 μg/ml, less than 60 μg/ml, less than 50 μg/ml, less than 40 μg/ml, less than 30 μg/ml, less than 20 μg/ml, less than 10 μg/ml, less than 5 μg/ml, less than 2.5 μg/ml, less than 2 μg/ml, less than 1 μg/ml, etc.).

The fluid module 200 and/or fluid circuit(s) connected to the fluid module 200 can optionally include one or more reservoirs. A reservoir can include a well and/or any other container. Examples of reservoirs include: liquid reservoirs (e.g., housing media components and/or other liquids from the fluid module 200), mixing reservoirs, disposal reservoirs (e.g., as part of a waste extraction system), sampling reservoirs (e.g., where samples of media can be deposited for measurement acquisition), media reservoirs (e.g., where media is housed for transport to the set of incubation modules 100 and/or components therein), and/or any other reservoir. In an example, a liquid reservoir can include a liquid sensor to measure mass and/or volume of liquid in the reservoir (e.g., to measure amount of liquid transported from the fluid module 200). In a first specific example, the liquid sensor can include a dielectric sensor (e.g., capacitive level sensing). In an illustrative example, the capacitance between two electrodes is measured relative to a reference electrode. In a second specific example, the liquid sensor can include a well plate scale beneath one or more liquid reservoirs.

In an example, a first reservoir connected to an inlet of a channel (e.g., a channel in a placental circuit) and a second reservoir connected to an outlet of the channel can function as pressure columns, controlling the flow through the channel. In a specific example, a fluid circuit (e.g., placental circuit), can include: a channel, a first reservoir connected to the inlet of the channel (e.g., where the first reservoir houses a first quantity of media), a second reservoir connected to the outlet of the channel (e.g., where the second reservoir houses a second quantity of the media), a first sensor (e.g., dielectric sensor) configured to measure the quantity of media in the first reservoir (e.g., the first quantity); and a second sensor (e.g., dielectric sensor) configured to measure the quantity of media in the second reservoir (e.g., the second quantity). The fluid module 200 can include a set of liquid handlers (e.g., pumps) configured to transport media into and/or out of the first reservoir and the second reservoir, based on the measured quantity (e.g., volume, mass, height, etc.) of liquid in each reservoir (e.g., and a target quantity for the first reservoir and a target quantity for the second reservoir). In a specific example, a first set of pumps (e.g., an inlet and outlet pump) can be configured to transport the media between the first reservoir and a third reservoir (e.g., housing a third quantity of media), and a second set of pumps (e.g., an inlet and outlet pump) can be configured to transport the media between the second reservoir and the third reservoir, wherein the first set of pumps and the second set of pumps can be controlled based on the measured quantity (e.g., volume, mass, height, etc.) of liquid in each reservoir. Examples are shown in FIG. 3 and FIG. 5.

The fluid module 200 can optionally include one or more liquid handlers. In a first variant, the liquid handler includes one or more pumps pressurizing liquid (e.g., media components) to transport the liquid. In a first example, a set of pumps can transport media (e.g., between reservoirs, from a reservoir into a container of an incubation module, through a fluid circuit, etc.). In a second example, a set of pumps (e.g., syringe drivers) can transport media components (e.g., hormones, cytokines, growth factors, etc.). In a specific example, the set of pumps can drive addition of one or more media components into a media reservoir. In an illustrative example, the system can include: a first media reservoir housing media for a placental circuit, a second media reservoir housing media for a uterine circuit (e.g., well circuit), a first set of pumps configured to deliver a first set of media components to the first media reservoir, and a second set of pumps configured to deliver a second set of media components to the second media reservoir. In a second variant, the liquid handler includes a pressure column (e.g., a media reservoir functions as a pressure column), wherein the media and/or other liquids in the pressure column can drive transport of the liquid to the set of containers via hydrostatic pressure. In a third variant, the liquid handler includes a robotic liquid handler. An example is shown in FIG. 8G. For example, the robotic liquid handler can change media in a container by performing one or more of: aspirating media components from liquid reservoir(s), dispensing the media components into a mixing reservoir, mixing the components to produce new media, aspirating old media (e.g., a portion of the old media) from the container (e.g., from the corner of the container to protect the biological specimen), dispensing the old media in a disposal reservoir and/or a sampling reservoir, aspirating the new media from the mixing reservoir, and dispensing the new media into the container. The chamber can optionally include one or more pipette tip containers housing pipette tips for a liquid handler. The chamber can optionally include one or more pipette tip disposal containers.

In an example, liquid can be transported using a media filler system and/or media bottle subsystem. In an example, a liquid can be transported from a liquid container (e.g., media bottle) within the fluid module 200 to the set of incubation modules 100 using pressurized gas (e.g., nitrogen, which can prevent or reduce oxidation of the liquid) from the gas module 250. In a specific example, transporting the liquid can include one or more of: controlling pressure of the gas using a variable pressure controller (e.g., optionally based on the viscosity of the liquid); providing the pressurized gas to the fluid module 200 via a valve (e.g., three-way valve); pressurizing a container housing the liquid via a solenoid manifold (e.g., controlled by a computing system), which moves the liquid through a liquid conduit into a liquid reservoir (e.g., a liquid reservoir within the chamber, via an interface in a wall of the chamber). The amount of liquid transported can optionally be monitored using a liquid sensor (e.g., well plate scale underneath the liquid reservoir). The amount of each liquid transported can be determined based on media parameters (e.g., determined via the processing system 400), sensor measurements (e.g., glucose measurements), and/or otherwise determined. A valve (e.g., the three-way valve, optionally connected to a vacuum pump) can optionally be opened to transport the liquid back into the liquid container (e.g., allowing pressure to vent, using a vacuum pump to extract the liquid from the conduit, etc.). The liquids can be transported in series and/or in parallel.

However, the fluid module 200 can be otherwise configured.

The gas module 250 functions to adjust gas parameter values (e.g., to adjust the oxygen saturation, to adjust the carbon dioxide saturation, etc.), to drive fluid transport, and/or for any other suitable functionalities). Examples of gas parameters values include: gas composition (e.g., oxygen saturation, carbon dioxide saturation), gas pressure, gas temperature, and/or any other parameter values. The gas module 250 can interface with the set of incubation modules 100, the fluid module 200, the sensor module 300, the chamber, and/or any other system component. The gas module 250 can include one or more gas control systems, where each gas control system controls gas parameter values in a container (e.g., a reservoir, an incubation module container, the chamber, etc.). In examples, the gas module 250 (e.g., each gas control system) can include one or more: gas canisters, gas conduits (e.g., from the canisters to the chamber and/or to the fluid module 200), mixers (e.g., a tri-gas mixer), oxygenators, compressors, filters, blowers, bubblers, valves, sensors (e.g., gas sensors, pH sensors, etc.), and/or any other system components. Examples of gasses can include oxygen, carbon dioxide, nitrogen, and/or any other gas. In an example, the gas module 250 can function to condition the media (e.g., before the media is supplied to the set of incubation modules 100, the placenta, the gel, and/or any fluid circuit). In a specific example, the gas module 250 includes an oxygenator, wherein media flows through the oxygenator to (re)oxygenate the media. In an illustrative example, media flows through a fluid circuit to supply oxygen and/or nutrients to the biological specimen; the media is then reoxygenated and recirculated through the fluid circuit. In a specific example, the media supplied to the maternal circuit can have a higher oxygen saturation than the media supplied to the uterine circuit.

In variants, the gas module 250 can include one or more gas control systems configured to control gas parameter values (e.g., composition, pressure, etc.) within one or more reservoirs and/or one or more gas control systems configured to control gas parameter values within all or a portion of one or more incubation modules in the set of incubation modules. In a specific example, the gas module 250 can include, for each media reservoir, a gas control system configured to control gas parameter values (e.g., composition, pressure, etc.) within the media reservoir. In another specific example, the gas module 250 can include, for each incubation module, a gas control system configured to control gas parameter values (e.g., composition, pressure, etc.) within all or a portion of the incubation module (e.g., within the container of the incubation module). In an example, a gas control system can be coupled to a reservoir, wherein the gas control system configured to control a composition of gas within the reservoir. In another example, a gas control system can be coupled to a container (e.g., well) in an incubation module, wherein the gas control system is configured to control a composition of gas within the container. In a specific example, the system can include: a first reservoir connected to the inlet of a placental circuit channel; a second reservoir connected to the outlet of the placental circuit channel; a first gas control system coupled to the first reservoir, the first gas control system configured to control a composition of gas within the first reservoir; and a second gas control system coupled to the second reservoir, the second gas control system configured to control a composition of gas within the second reservoir; and, optionally, a third gas control system coupled to a container (e.g., well) in an incubation module, the third gas control system configured to control a composition of gas within the container.

However, the gas module 250 can be otherwise configured.

The system can optionally include one or more environmental control components, which can function to control the environment experienced by the set of biological specimens according to a set of environmental parameters (e.g., determined by the processing system 400). Environmental control components can include one or more of: the gas module 250 and/or components therein, heater system, humidifier, actuators (e.g., to vibrate and/or otherwise actuate an incubation module), electrodes (e.g., to provide electrical stimulation to a biological specimen), computing system, and/or any other components. Examples of gas systems (e.g., tri gas mixer) are shown in FIG. 8H and FIG. 8I. An example of a heater system is shown in FIG. 8C. However, environmental control component(s) can be otherwise configured.

The sensor module 300 functions to: perform error checks, collect measurements of the set of biological specimens, collect measurements of liquids (e.g., media), collect measurements of gas, collect measurements of the environment in the chamber, and/or collect any other data. Measurements can be collected in responsive to a request, iteratively, concurrently, asynchronously, periodically (e.g., at least every 2 hours, at least every 1 hour, at least every 30 min, at least every 15 min, etc., and/or at any other suitable time.

In examples, the sensor module 300 can include one or more sensors located: above the set of biological specimens, on the side of the set of biological specimens, below the set of biological specimens, in a sampling reservoir and/or any other reservoir, in or near the set of incubation modules 100, at a gas mixer, a combination thereof, and/or any other location relative to the set of incubation modules 100. The sensor module 300 and/or components therein can optionally be actuated (e.g., in 1 dimension, in 2 dimensions, 3 dimensions, etc.). In a specific example, a camera and/or a light source can each be actuated in 1, 2, or 3 dimensions (e.g., on a gantry, on an x, xy, or xyz translation stage, etc.). Actuation can optionally be controlled by the processing system 400. In an example, the sensor module 300 can include an imaging system (e.g., including a camera and/or light source) that can be actuated to collect images of each of the set of biological specimens. Examples of sensors in the sensor module 300 can include: an imaging device (e.g., camera, detector, light source, image sensor, etc.), depth sensor, thermal sensor, humidity, pH, gas sensor (e.g., CO2, O2, nitrogen, etc.), glucose sensor, lactic acid, urea sensor, ultrasound, sensor for any media component, heartbeat sensor (e.g., piezo-based contact microphone, MEMS hydrophone, electrodes, etc.; specific to an incubation module and/or global across incubation modules), electrodes, cell sequencers, and/or any other sensor.

The imaging device can measure infrared, visible light (e.g., RBG, red light, etc.), and/or any other wavelength. The imaging device can optionally include a microscopy system (e.g., an optical microscopy system that includes a camera). In examples, the microscopy system can use transmission microscopy, reflected light microscopy, fluorescence microscopy, infinity corrected optical systems, phase contrast microscopy, light sheet microscopy, and/or any other microscopy methods. In an example, the imaging system can include a light sheet microscope. In a specific example, the light sheet microscope can collect a set of images in a set of planes intersecting with the biological specimen. In a specific example, a three-dimensional (3D) model of the biological specimen can be reconstructed from the set of images (e.g., including segmenting the biological specimen in the set of images). In an example, a 3D model of a biological specimen (e.g., each biological specimen in the set of biological specimens) can be determined based on a set of images collected by the imaging device, wherein the developmental stage of the biological specimen is determined based on the 3D model. The imaging device can optionally include one or more light sources (e.g., lamp, LEDs, laser, etc.). The light source can output infrared, visible light (e.g., RBG, red light, etc.), and/or any other wavelength. In examples, the light source can be top-down (e.g., mounted to an actuated camera), bottom-up (e.g., illuminating from below the set of incubation modules 100), side-on (e.g., illuminating from a side of the set of incubation modules 100), ambient light, a combination thereof, and/or any other light source location. The light source and camera of the imaging device can optionally be orthogonal to one another. In a first example, the light source can be top-down, and the camera can be side-on. In a second example, the light source can be side-on and the camera can be top-down. The imaging device module can optionally include diffusers, filters, lenses, and/or any other optics components.

The sensor module 300 can collect data periodically (e.g., at predetermined regular or irregular intervals such as every 1 hr-24 hr, every 6 hr, etc.), upon request, upon a trigger from data acquired by another sensor, and/or at any other time. In a first example, the sensor module 300 can image the biological specimen to monitor development. In a second example, the sensor module 300 can measure levels of glucose, lactic acid, urea, and/or other media components to monitor metabolism. In a third example, the sensor module 300 can measure fluorescence and/or other markers to demonstrate that placentation and/or perfusion of the placenta has occurred. In a first illustrative example, placenta cells can be genetically engineered to include fluorescent markers; the sensor module 300 can be used to track growth of the placenta by tracking the fluorescence. In a second illustrative example, media can include fluorescent dye; the sensor module 300 can be used to track the fluorescent dye flowing through the incubation module (e.g., through a fluid circuit), through placental tissue of the biological specimen, into the heart of the biological specimen, and/or throughout the biological specimen body. In a fourth example, the sensor module 300 can perform (destructive and/or nondestructive) sequencing of a biological specimen, which can be used to adjust parameters for the other biological specimens in the set.

The processing system 400 functions to receive measurements, process measurements, and/or control one or more of: the set of incubation modules 100, the chamber, the fluid module 200 (e.g., one or more pumps in the fluid module 200), the gas module 250 (e.g., one or more gas control systems in the gas module 250), environmental control components, and/or any other system components. In variants, the processing system 400 can function to control one or more system components (e.g., the set of incubation modules 100, the chamber, the fluid module 200, the gas module 250, environmental control components, etc.) based on measurements received from the sensor module 300. For example, the processing system 400 can function to control the incubation protocol (e.g., gestation protocol) for the set of biological specimens by adjusting parameter values (e.g., incubation parameter values). The processing system 400 can be a component of and/or communicate with the set of incubation modules 100, the chamber, the fluid module 200, the gas module 250, environmental control components, and/or any other components.

The processing system 400 can include one or more: CPUs, GPUs, TPUs, custom FPGA/ASICS, microprocessors, servers, cloud computing, and/or any other suitable components. The processing system 400 can be local, remote, distributed, and/or otherwise arranged relative to any other system or module. The processing system 400 can optionally include and/or interface with a database (e.g., storing and/or retrieving measurements, parameter values, etc.).

The processing system 400 can include or use one or more models, including a developmental stage model, a protocol model, and/or any other model. The models can include classical or traditional approaches, machine learning approaches, and/or be otherwise configured. The models can include regression, decision tree, clustering, association rules, dimensionality reduction, neural networks (e.g., GNN, CNN, DNN, CAN, LSTM, RNN, FNN, encoders, decoders, deep learning models, transformers, etc.), ensemble methods, optimization methods, cost functions, classification, rules, heuristics, equations (e.g., weighted equations, etc.), selection (e.g., from a library), lookups, regularization methods, Bayesian methods, instance-based methods, kernel methods, support vectors, statistical methods (e.g., probability), comparison methods (e.g., distance metrics, thresholds, etc.), deterministics, genetic programs, foundation models, and/or any other suitable model. Models can be trained, learned, fit, predetermined, and/or can be otherwise determined. The models can be trained or learned using: supervised learning, unsupervised learning, self-supervised learning, semi-supervised learning, reinforcement learning, transfer learning, Bayesian optimization, fitting, interpolation and/or approximation, backpropagation, and/or otherwise generated. The models can be learned or trained on: labeled data, unlabeled data, positive training sets, negative training sets, and/or any other suitable set of data.

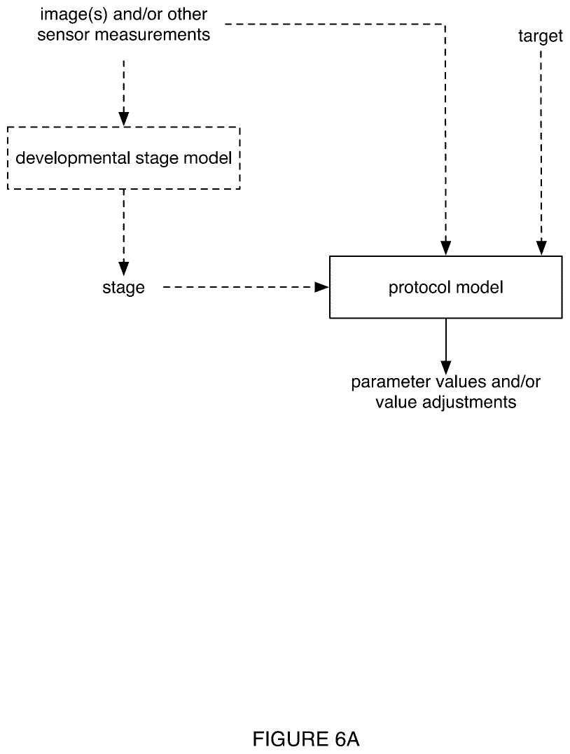

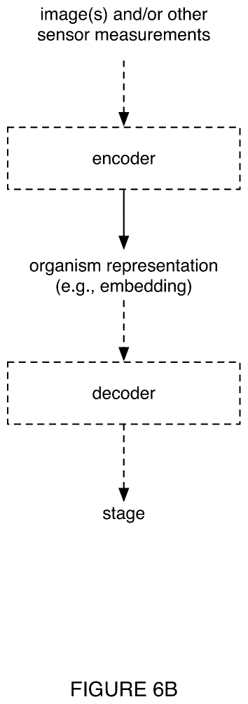

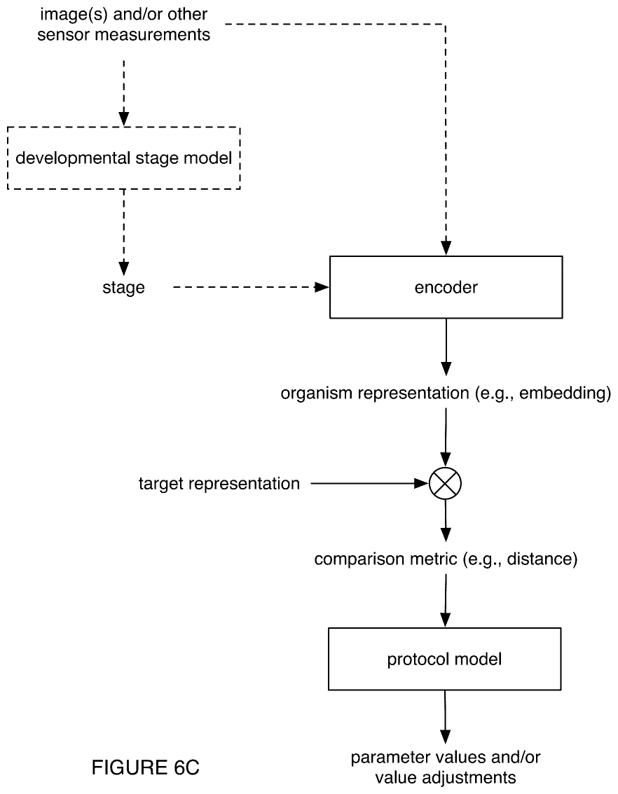

The processing system 400 can optionally classify the developmental stage of a biological specimen using a developmental stage model. Examples are shown in FIG. 6A, FIG. 6B, and FIG. 6C. Developmental stages can optionally be aggregated (e.g., averaged) across the set of biological specimens. Inputs to the developmental stage model can include: one or more images of the biological specimen, a 3D model of the biological specimen (e.g., segmented from the one or more images), other sensor measurements, time (e.g., relative to the start of incubation of the biological specimen), and/or any other suitable inputs. Outputs from the developmental stage model can include one or more: the developmental stage (e.g., Theiler Stage), confidence level associated with the developmental stage, and/or other suitable outputs. In an illustrative example, the developmental stage model classifies an embryo as: 0% probability TS1; 0% probability TS2; 10% probability TS3; 80% probability TS4; 10% probability TS5; and 0% probability TS6-TS28. In examples, the developmental stage model can include a CNN (e.g., deep CNN), a classifier, computer vision and/or other image processing methods, and/or any other models. In an example, determining the developmental stage of the embryo can include: generating an embedding for the embryo based on a set of images and optionally any other inputs, and determining the developmental stage based on the embedding. In a specific example, the developmental stage model can include: an first model (e.g., including an encoder) configured to output an embedding (e.g., latent vector) for the biological specimen based on the image(s) of the biological specimen and optionally any other inputs; and a second model (e.g., including a decoder) configured to output the developmental stage based on the embedding.

In a first example, the developmental stage model can be trained using training data collected by: explanting biological specimens (e.g., embryos) gestated in-vivo, imaging the biological specimens (e.g., using the sensor module 300 and/or a separate microscopy system), and labeling the training images with their associated developmental stage. In a second example, the developmental stage model can be trained using training data collected by: imaging biological specimens (e.g., embryos) while gestating in-vivo (e.g., using a μCT system), and labeling the training images with their associated developmental stage. In a third example, the developmental stage model can be trained using training data collected by imaging biological specimens while incubating in the system, and labeling the training images with their associated developmental stage. A generative model (e.g., using a Generative Adversarial Network (GAN) architecture) can optionally be used to generate synthetic images and/or other synthetic data for an artificial biological specimen. This synthetic data can optionally be used for further investigation of the developmental stages and/or to augment the training dataset.

The processing system 400 can control values for one or more parameters, including environmental parameters (e.g., via environmental control components, via the gas module 250, etc.), media parameters (e.g., via the fluid module 200, via the liquid handler, etc.), and/or any other parameters. Environmental parameters can include temperature, humidity, pressure, gas composition (e.g., percent O2, percent CO2, etc.), electrode stimulation, actuation (e.g., pressurization, vibration, etc.), any other gas parameters, and/or other environmental parameters. Media parameters can include flow rate, media change timing, media composition, media oxygenation, media volume, and/or other media parameters.

The parameter values can be predetermined, manually determined, automatically determined (e.g., output by a protocol model), randomly determined, determined based on one or more measurements, determined based on a developmental stage, determined based on time, a combination thereof, and/or otherwise determined. For example, the processing system 400 can adjust the parameter values (e.g., in real time) based on one or more measurements acquired via the sensor module 300 (e.g., a control loop with multimodal inputs). The processing system 400 can update parameter values continuously, periodically (e.g., predetermined regular or irregular intervals), upon request, based on a trigger (e.g., when data acquired by a sensor crosses a threshold, when a biological specimen reaches the next developmental stage, when a biological specimen is not developing, etc.), and/or at any other time. In an example, the processing system can be configured to: determine a developmental stage of the biological specimen (e.g., embryo) based on a set of measurements (e.g., images of the biological specimen); based on the developmental stage of the embryo, control the fluid module 200 (e.g., one or more pumps); and based on the developmental stage of the embryo, control the gas module 250 (e.g., one or more gas control systems).

The processing system 400 can optionally halt and/or reduce all or a portion of support and/or any other interaction with a biological specimen (e.g., stop media changes) if the biological specimen is not developing appropriately (e.g., not developing fast enough, not developing at a similar pace to the other biological specimens in the set, etc.). In variants, this can preserve resources and/or increase efficiency of the system.

In a first variant, the processing system 400 can control all or a subset of the parameter values to follow a predetermined incubation protocol. In a first embodiment, the predetermined incubation protocol includes constant values maintained throughout incubation. For example, the temperature of the chamber can be maintained between at or near 37° C. In another example, humidity of the chamber can be maintained between 80%-100% or any range or value therebetween (e.g., at least 85%). In a second embodiment, the predetermined incubation protocol includes multiple protocol phases, each phase associated with: a set of parameter values, a rate of range of parameter values, and/or any other parameter settings. For example, the processing system 400 can advance to the next protocol phase based on developmental stage classification, time, manually, and/or any other trigger.

In a second variant, the processing system 400 can control all or a subset of the parameter values based on an output from a protocol model. Examples are shown in FIG. 6A and FIG. 6C. Inputs to the protocol model can include: sensor measurements, developmental stage and/or associated confidence levels for one or more of the set of biological specimens, an embedding for a biological specimen (e.g., determined using the developmental stage model), current and/or previous parameter values, time, and/or any other inputs. Outputs from the protocol model can include: new parameter values, parameter value adjustments (e.g., relative to current parameter values), and/or any other outputs. All or a subset of the outputs can be specific to a biological specimen and/or global across biological specimens. In a specific example, outputs can include parameter values specific to each biological specimen in the set of biological specimens, wherein the outputs can optionally be aggregated (e.g., averaged) to generate global parameter values. In a specific example, the protocol model can be a deep reinforcement learning model. In an example, determining updated (e.g., optimized) parameter values can include: inputting the developmental stage of a biological specimen and/or sensor measurements into an encoder to generate a representation for the biological specimen (e.g., embedding, latent vector such as a low-dimensional dense vector, etc.); determining a comparison metric (e.g., a distance) between the representation and a target representation; inputting the comparison metric into the protocol model to generate the updated parameter values for the biological specimen. In a specific example, the protocol model can be or include a cost function.

In an example, the target representation can be determined (e.g., learned) by: explanting embryos at multiple stages of intrauterine gestation, performing measurements of the explanted biological specimens, using the encoder to generate a reference representation of each explanted embryo, and determining a target representation for each developmental stage based on the reference representations (e.g., aggregating reference representations that correspond to the same developmental stage, interpolating between reference representations corresponding to different developmental stages, determining target representations as a function of developmental stage using a regression and/or other models, etc.). The measurements of the explanted embryo are preferably performed in the chamber using the sensor module 300, but can additionally or alternatively be otherwise performed (e.g., imaging using a separate microscopy system).

The protocol model can optionally be trained using augmented training data. For example, the training data can be augmented with synthetic data (e.g., synthetic images, synthetic data generated from a digital twin, etc.). In a specific example, synthetic training representations can be determined by interpolating between training representations of training biological specimens, wherein the synthetic training representations can be directly used as synthetic training data and/or can be decoded to determine synthetic training sensor measurements (e.g., images). In an illustrative example, a smooth continuous embedding space between TS0 and TS28 (for target biological specimens and/or failure modes) can be determined using target and/or failure training biological specimens, wherein synthetic training data for artificial biological specimen at any development stage can be generated by selecting a point in the embedding space. Training the protocol model can optionally include training the protocol model for a first species (e.g., mice), and retraining (e.g., fine-tuning) the protocol model for another species (e.g., sequentially moving to species closer to humans).

In an illustrative example, a tri gas mixer (TGM) can be controlled using the protocol model (e.g., including a cost function). For example, the TGM can have CO₂, N, and O2 as inputs. The input flow can be controlled by a proportional valve that can be set continuously between 0% and 100%. The TGM can measure the CO 2 and O 2 percentage by volume in the enclosure and infers N2 using yₙ_{₂}(t)=1−y_{co}_{₂}(t)−yₒ_{₂}(t). A cost function can be used to minimize distance away from the setpoints and/or minimizing the total output (e.g., to minimize gas usage). Introducing notation such that r is the set point, y is process value, and u is the control variable and e=r−y is the error, the following cost function can be minimized: C(t)=(y_{co}_{₂}(t)−r_{co}_{₂}(t))²+(yₙ_{₂}(t)−rₙ_{₂}(t))²+(yₒ_{₂}(t)−rₒ_{₂}(t))²+λ(u_{co}_{₂}(t)+uₙ_{₂}(t)+uₒ_{₂}(t)). In a specific example, the state of the system can be given by: {y_{co}_{₂}(t), yₒ_{₂}(t), u_{co}_{₂}(t), uₙ_{₂}(t), uₒ_{₂}(t)} The TGM output at the next time step can be calculated using: {u_{co}_{₂}(t+dt), uₙ_{₂}(t+dt), uₒ_{₂}(t+dt)}.

However, the processing system 400 can be otherwise configured.

All or a portion of one or more biological specimens in the set of biological specimens can optionally be extracted (e.g., harvested) after incubation (e.g., after development). For example, cells in a biological specimen (e.g., embryo) can be extracted for use in a therapeutic. In a first variant, extracting all or a portion of a biological specimen can include: disassociating cells of the biological specimen, sorting the cells, and extracting a portion of the cells (e.g., one or more cell types). In a second variant, extracting all or a portion of a biological specimen can include: dissecting one or more tissues (e.g., microdissection, using laser ablation) from the biological specimen, and optionally maturing the one or more tissues (e.g., in an extra culture stage). However, extracting all or a portion of the biological specimen(s) can be otherwise performed.

However, the system can be otherwise configured.

### 4. Specific Examples

A numbered list of specific examples of the technology described herein are provided below. A person of skill in the art will recognize that the scope of the technology is not limited to and/or by these specific examples.

Specific Example 1. A system for developing an embryo, the system comprising: a well having a base surface defining a first opening, wherein the embryo is housed within the well; a gel, wherein the gel is disposed between the embryo and the base surface of the well; a first fluid circuit configured to supply a first media to the well; and a second fluid circuit comprising a channel coupled to the base surface of the well, wherein a second media passes through the channel, the channel comprising a top surface defining a second opening, wherein the first opening at least partially overlaps with the second opening.

Specific Example 2. The system of Specific Example 1, wherein the channel comprises an inlet and an outlet, wherein the second fluid circuit further comprises: a first reservoir connected to the inlet of the channel, the first reservoir housing a first quantity of the second media; a second reservoir connected to the outlet of the channel, the second reservoir housing a second quantity of the second media; a first sensor configured to measure the first quantity of the second media; a second sensor configured to measure the second quantity of the second media; a first set of pumps configured to transport the second media between the first reservoir and a third reservoir, the third reservoir housing a third quantity of the second media; and a second set of pumps configured to transport the second media between the second reservoir and the third reservoir; the system further comprising a processing system 400 configured to control the first set of pumps and the second set of pumps based on measurements received from the first sensor and the second sensor.

Specific Example 3. The system of Specific Example 2, further comprising a set of gas control systems comprising: a first gas control system coupled to the first reservoir, the first gas control system configured to control a composition of gas within the first reservoir; a second gas control system coupled to the second reservoir, the second gas control system configured to control a composition of gas within the second reservoir; and a third gas control system coupled to the well, the third gas control system configured to control a composition of gas within the well.

Specific Example 4. The system of Specific Example 3, wherein the first fluid circuit is connected to a fourth reservoir housing a quantity of the first media, the system further comprising: a third set of pumps configured to deliver a first set of media components to the third reservoir; a fourth set of pumps configured to deliver a second set of media components to the fourth reservoir; an imaging system configured to collect a set of images of the embryo; and a processing system 400 configured to, based on the set of images, control the set of gas control systems, the third set of pumps, and the fourth set of pumps.

Specific Example 5. The system of Specific Example 4, wherein the processing system 400 is configured to determine a developmental stage of the embryo based on the set of images, wherein the set of gas control systems, the third set of pumps, and the fourth set of pumps are each controlled based on the developmental stage.

Specific Example 6. The system of any of Specific Examples 4-5, wherein the imaging system comprises a light sheet microscope.

Specific Example 7. The system of any of Specific Examples 2-6, wherein the first sensor and the second sensor each comprise a dielectric sensor.

Specific Example 8. The system of any of Specific Examples 1-7, wherein a second embryo is housed within the well.

Specific Example 9. The system of any of Specific Example 1-8, wherein the embryo comprises a mouse embryo or a human embryo.

Specific Example 10. The system of any of Specific Examples 1-9, wherein cells in the embryo are extracted for use in a therapeutic.

Specific Example 11. A system for developing a set of embryos, the system comprising: a first reservoir housing a first media; a set of pumps configured to deliver a set of media components to the first reservoir; a set of incubation modules, wherein incubation module is configured to house an embryo in the set of embryos, wherein each incubation module comprises: a well having a base surface, wherein an embryo in the set of embryos is housed within the well; a gel, wherein the gel is disposed between the embryo and the base surface of the well; and a first fluid circuit connected to the first reservoir, the first fluid circuit configured to supply the first media to the well; a gas control system configured to control a composition of gas within the well of each incubation module; an imaging device configured to collect a set of images of each embryo in the set of incubation modules; a processing system 400 configured to: using a model, determine a developmental stage of the embryo based on the set of images; based on the developmental stage of the embryo, control the set of pumps; and based on the developmental stage of the embryo, control the gas control system.

Specific Example 12. The system of Specific Example 11, wherein the model comprises a machine learning model, wherein determining the developmental stage of the embryo comprises: generating an embedding for the embryo based on the set of images, and determining the developmental stage based on the embedding.

Specific Example 13. The system of any of Specific Examples 11-12, wherein the imaging system comprises a light sheet microscope.

Specific Example 14. The system of Specific Example 13, wherein the light sheet microscope is configured to collect the set of images using infrared light.

Specific Example 15. The system of any of Specific Examples 13-14, wherein a three-dimensional model of each embryo is determined based on the set of images, wherein the developmental stage of the embryo is determined based on the three-dimensional model.

Specific Example 16. The system of any of Specific Examples 11-15, wherein each incubation module further comprises a second fluid circuit comprising a channel coupled to the base surface of the well, wherein the base surface of the well defines a first opening, wherein a second media passes through the channel, the channel comprising a top surface defining a second opening, wherein the first opening at least partially overlaps with the second opening.

Specific Example 17. The system of Specific Example 16, wherein, for each incubation module, the channel comprises an inlet and an outlet, wherein the second fluid circuit further comprises: a second reservoir connected to the inlet of the channel, the second reservoir housing a first quantity of the second media; a third reservoir connected to the outlet of the channel, the third reservoir housing a second quantity of the second media; a first sensor configured to measure the first quantity of the second media; a second sensor configured to measure the second quantity of the second media; a first set of pumps configured to transport the second media between the second reservoir and a fourth reservoir, the fourth reservoir housing a third quantity of the second media; and a second set of pumps configured to transport the second media between the third reservoir and the fourth reservoir; wherein the processing system 400 is further configured to control the first set of pumps and the second set of pumps based on measurements received from the first sensor and the second sensor.

Specific Example 18. The system of any of Specific Examples 11-17, wherein the set of pumps comprise syringe drivers.

Specific Example 19. The system of any of Specific Examples 11-18, wherein the set of embryos comprises a mouse embryo or a human embryo.

Specific Example 20. The system of any of Specific Examples 11-19, wherein cells in the embryo are extracted for use in a therapeutic.

All references cited herein are incorporated by reference in their entirety, except to the extent that the incorporated material is inconsistent with the express disclosure herein, in which case the language in this disclosure controls.

As used herein, “substantially” or other words of approximation can be within a predetermined error threshold or tolerance of a metric, component, or other reference, and/or be otherwise interpreted.

Optional elements, which can be included in some variants but not others, are indicated in broken line in the figures.

Different subsystems and/or modules discussed above can be operated and controlled by the same or different entities. In the latter variants, different subsystems can communicate via: APIs (e.g., using API requests and responses, API keys, etc.), requests, and/or other communication channels. Communications between systems can be encrypted (e.g., using symmetric or asymmetric keys), signed, and/or otherwise authenticated or authorized.

Alternative embodiments implement the above methods and/or processing modules in non-transitory computer-readable media, storing computer-readable instructions that, when executed by a processing system, cause the processing system to perform the method(s) discussed herein. The instructions can be executed by computer-executable components integrated with the computer-readable medium and/or processing system. The computer-readable medium may include any suitable computer readable media such as RAMs, ROMs, flash memory, EEPROMs, optical devices (CD or DVD), hard drives, floppy drives, non-transitory computer readable media, or any suitable device. The computer-executable component can include a computing system and/or processing system (e.g., including one or more collocated or distributed, remote or local processors) connected to the non-transitory computer-readable medium, such as CPUs, GPUs, TPUS, microprocessors, or ASICs, but the instructions can alternatively or additionally be executed by any suitable dedicated hardware device.

Embodiments of the system and/or method can include every combination and permutation of the various system components and the various method processes, wherein one or more instances of the method and/or processes described herein can be performed asynchronously (e.g., sequentially), contemporaneously (e.g., concurrently, in parallel, etc.), or in any other suitable order by and/or using one or more instances of the systems, elements, and/or entities described herein. Components and/or processes of the following system and/or method can be used with, in addition to, in lieu of, or otherwise integrated with all or a portion of the systems and/or methods disclosed in the applications mentioned above, each of which are incorporated in their entirety by this reference.

As a person skilled in the art will recognize from the previous detailed description and from the figures and claims, modifications and changes can be made to the preferred embodiments of the invention without departing from the scope of this invention defined in the following claims.

We claim:

1. A system for developing an embryo, the system comprising:
a well having a base surface defining a first opening, wherein the embryo is housed within the well;
a gel, wherein the gel is disposed between the embryo and the base surface of the well;
a first fluid circuit configured to supply a first media to the well; and
a second fluid circuit comprising a channel coupled to the base surface of the well, wherein a second media passes through the channel, the channel comprising a top surface defining a second opening, wherein the first opening at least partially overlaps with the second opening.

2. The system of claim 1, wherein the channel comprises an inlet and an outlet, wherein the second fluid circuit further comprises:
a first reservoir connected to the inlet of the channel, the first reservoir housing a first quantity of the second media;
a second reservoir connected to the outlet of the channel, the second reservoir housing a second quantity of the second media;
a first sensor configured to measure the first quantity of the second media;
a second sensor configured to measure the second quantity of the second media;
a first set of pumps configured to transport the second media between the first reservoir and a third reservoir, the third reservoir housing a third quantity of the second media; and
a second set of pumps configured to transport the second media between the second reservoir and the third reservoir;
the system further comprising a processing system configured to control the first set of pumps and the second set of pumps based on measurements received from the first sensor and the second sensor.

3. The system of claim 2, further comprising a set of gas control systems comprising:
a first gas control system coupled to the first reservoir, the first gas control system configured to control a composition of gas within the first reservoir;
a second gas control system coupled to the second reservoir, the second gas control system configured to control a composition of gas within the second reservoir; and
a third gas control system coupled to the well, the third gas control system configured to control a composition of gas within the well.

4. The system of claim 3, wherein the first fluid circuit is connected to a fourth reservoir housing a quantity of the first media, the system further comprising:
a third set of pumps configured to deliver a first set of media components to the third reservoir;
a fourth set of pumps configured to deliver a second set of media components to the fourth reservoir;
an imaging system configured to collect a set of images of the embryo; and
a processing system configured to, based on the set of images, control the set of gas control systems, the third set of pumps, and the fourth set of pumps.

5. The system of claim 4, wherein the processing system is configured to determine a developmental stage of the embryo based on the set of images, wherein the set of gas control systems, the third set of pumps, and the fourth set of pumps are each controlled based on the developmental stage.

6. The system of claim 4, wherein the imaging system comprises a light sheet microscope.

7. The system of claim 2, wherein the first sensor and the second sensor each comprise a dielectric sensor.

8. The system of claim 1, wherein a second embryo is housed within the well.

9. The system of claim 1, wherein the embryo comprises a mouse embryo or a human embryo.

10. The system of claim 1, wherein cells in the embryo are extracted for use in a therapeutic.

11. A system for developing a set of embryos, the system comprising:
a first reservoir housing a first media;
a set of pumps configured to deliver a set of media components to the first reservoir;
a set of incubation modules, wherein incubation module is configured to house an embryo in the set of embryos, wherein each incubation module comprises:
a well having a base surface, wherein an embryo in the set of embryos is housed within the well;
a gel, wherein the gel is disposed between the embryo and the base surface of the well; and
a first fluid circuit connected to the first reservoir, the first fluid circuit configured to supply the first media to the well;
a gas control system configured to control a composition of gas within the well of each incubation module;
an imaging device configured to collect a set of images of each embryo in the set of incubation modules;
a processing system configured to:
using a model, determine a developmental stage of the embryo based on the set of images;
based on the developmental stage of the embryo, control the set of pumps; and
based on the developmental stage of the embryo, control the gas control system.

12. The system of claim 11, wherein the model comprises a machine learning model, wherein determining the developmental stage of the embryo comprises: generating an embedding for the embryo based on the set of images, and determining the developmental stage based on the embedding.

13. The system of claim 11, wherein the imaging system comprises a light sheet microscope.

14. The system of claim 13, wherein the light sheet microscope is configured to collect the set of images using infrared light.

15. The system of claim 13, wherein a three-dimensional model of each embryo is determined based on the set of images, wherein the developmental stage of the embryo is determined based on the three-dimensional model.

16. The system of claim 11, wherein each incubation module further comprises a second fluid circuit comprising a channel coupled to the base surface of the well, wherein the base surface of the well defines a first opening, wherein a second media passes through the channel, the channel comprising a top surface defining a second opening, wherein the first opening at least partially overlaps with the second opening.

17. The system of claim 16, wherein, for each incubation module, the channel comprises an inlet and an outlet, wherein the second fluid circuit further comprises:
a second reservoir connected to the inlet of the channel, the second reservoir housing a first quantity of the second media;
a third reservoir connected to the outlet of the channel, the third reservoir housing a second quantity of the second media;
a first sensor configured to measure the first quantity of the second media;
a second sensor configured to measure the second quantity of the second media;
a first set of pumps configured to transport the second media between the second reservoir and a fourth reservoir, the fourth reservoir housing a third quantity of the second media; and
a second set of pumps configured to transport the second media between the third reservoir and the fourth reservoir;
wherein the processing system is further configured to control the first set of pumps and the second set of pumps based on measurements received from the first sensor and the second sensor.

18. The system of claim 11, wherein the set of pumps comprise syringe drivers.

19. The system of claim 11, wherein the set of embryos comprises a mouse embryo or a human embryo.

20. The system of claim 11, wherein cells in the embryo are extracted for use in a therapeutic.
